(19) United States
(12) Patent Application Publication
(10) Pub. No.: US 2018/0128801 A1
(43) Pub. Date: May 10, 2018

(54) HUMIDITY SENSOR AND METHOD OF EVALUATION

(71) Applicant: Robert Bosch GmbH, Stuttgart (DE)

(72) Inventors: Daniel Koenig, Leinfelden-Echterdingen (DE); Erick Gonzalez Rodriguez, Reutlingen (DE); Gabor Klimaj, Budapest (HU); Peter Bakucz, Klosterlechfeld (DE); Roberto Ferrozzi, Reutlingen (DE)

(21) Appl. No.: 15/804,492

(22) Filed: Nov. 6, 2017

(30) Foreign Application Priority Data
Nov. 7, 2016 (DE) 102016221742.9

(51) Int. Cl. G01N 33/00

(52) U.S. Cl. CPC G01N 33/0062; G01N 33/0036

(57) ABSTRACT

A humidity sensor having a housing, a data logger that has such a humidity sensor as well as a evaluation method for the humidity sensor or the data logger. The housing of the humidity sensor has a recess in which a humidity sensor element is situated. To protect the humidity sensor element against the ingress of water, a membrane is provided that covers at least a portion of the recess and thus defines a detection volume around the humidity sensor element. This membrane allows for an exchange of air with the surroundings so that the humidity sensor element is able to detect the air humidity. It is also designed in such a way that it prevents the ingress of water. The detection volume is designed to be as small so that the humidity of the detection volume adapts quickly to the surrounding climate that is to be measured.

### CROSS REFERENCE

The present application claims the benefit under 35 U.S.C. § 119 of German Patent Application No. DE 102016221742.9 filed on Nov. 7, 2016, which is expressly incorporated herein by reference in its entirety.

### FIELD

The present invention relates to a humidity sensor as well as to a data logger for information about the air humidity content and to a corresponding evaluation method.

### BACKGROUND INFORMATION

To detect the climatic conditions during the transport of perishable goods, e.g., foodstuffs, data loggers are used to detect and store the transport conditions. In a direct connection or an open housing construction of the corresponding humidity sensor with respect to the surroundings, there may be the problem that condensation situations or spray water during transport may constitute a falsification of the measured values or even an increased risk of failure of the sensor. As a remedy, humidity sensors in immersion-proof/splash-proof housings are used, whose interior climate is connected to the surrounding climate via a membrane that is permeable to air and permeable to humidity. Normally, however, this drastically reduces the reaction time of the connected data logger, since the humidity exchange through the membrane occurs markedly more slowly than in the case of a direct connection to the surroundings. Moreover, the incoming humidity may be absorbed by the utilized polymer materials of the data logger such that a humidity-reducing effect on the interior volume may be ascertained.

As a result, it is no longer possible to detect condensation situations and high relative humidities, which may result in damage to the transport goods and which may also occur within a short time due to high temperature fluctuations during transport. This results in undiscovered damage situations and in the fact that the data logger is unable to fulfill its purpose in said situations.

### SUMMARY

The present invention is directed to a humidity sensor having a housing, a data logger that has such a humidity sensor as well as an evaluation method for the humidity sensor or the data logger.

The housing of the humidity sensor has a recess in which a humidity sensor element is situated. In order to protect the humidity sensor element against the ingress of water, a membrane is provided that covers at least a portion of the recess and thus defines a detection volume around the humidity sensor element. This membrane is designed in such a way that it allows for an exchange of air with the surroundings so that the humidity sensor element is able to detect the air humidity. It is also designed in such a way, however, that it essentially prevents the ingress of water. The detection volume is furthermore designed to be as small as possible so that the humidity of the detection volume adapts as quickly as possible to the surrounding climate that is to be measured.

The walls of the recess or the detection volume may be made of metal so that it is possible to exclude an absorption of humidity on the part of the walls and thus an influence on the climate of the detection volume. Instead of a complete design of the housing from metal, it is also possible to provide that only the wall is covered with a metal layer. Alternatively, another material may also be used specifically for the wall of the recess that exhibits no or only very little absorption of humidity from the air.

In accordance with the present invention, the detection volume is consciously designed to be very small so that it is possible for the humidity of the detection volume to adapt to the surroundings quickly. For this purpose, the present invention provides for the detection volume to be dimensioned in such a way that the humidity sensor element takes up a substantial portion of the volume. In this connection it may be particularly provided that the humidity sensor element takes up at least a quarter of the detection volume, which is enclosed by the walls of the recess, the membrane and possibly a mount on which the humidity sensor element is mounted.

A design of the recess as a cylinder, starting from the surface of the housing, is particularly advantageous. In this instance, the membrane is provided on the upper end of the cylinder in the area of the surface of the housing. The humidity sensor element is in this instance mounted on the wall or on the bottom of the cylinder. Positioning may occur via a mounting board (normally a circuit board), on which the humidity sensor element is mounted. This mounting board may be connected to the walls of the cylindrical recess, e.g., via a seal in the form of a sealing ring. Alternative seals are also possible, however.

Furthermore, an evaluation unit may be provided that is advantageously located in the interior of the housing so that this evaluation unit is protected against external environmental influences. The evaluation unit is advantageously situated on the same mounting board as the humidity sensor element.

In the corresponding evaluation method, mutually associated temperature and humidity (sensor) variables are detected, using which it is possible to determine the air humidity. This determination is advantageously carried out at different times using respectively newly detected temperature/humidity variables. For this purpose, it is possible for example to use also the change in the temperature/humidity variables in order to ascertain the air humidity.

The data logger for the humidity sensor is designed in such a way that it stores the detected air humidity or the associated air humidity variables for later inspection or processing in a corresponding internal or external memory. Furthermore, it is also possible, however, for these air humidity variables to be transmitted to a corresponding indicator. It is furthermore possible for the individual or cumulated air humidity variables to be compared to one or multiple threshold values. If it is determined in the process that the air humidity at a point in time or over a specified time period is above a threshold value, then a corresponding warning signal may be generated or stored. This warning signal may also be indicated via an indicator.

Further advantages result from the description below of exemplary embodiments and the figures.

### BRIEF DESCRIPTION OF THE DRAWINGS

FIG. 1 schematically shows a system for ascertaining an air humidity variable based on detected temperature/humidity variables.

FIG. 2 shows one possible example construction of the present.

FIG. 3 shows a possible development of an evaluation method for the present invention with the aid of a flow chart.

### DETAILED DESCRIPTION OF EXAMPLE EMBODIMENTS

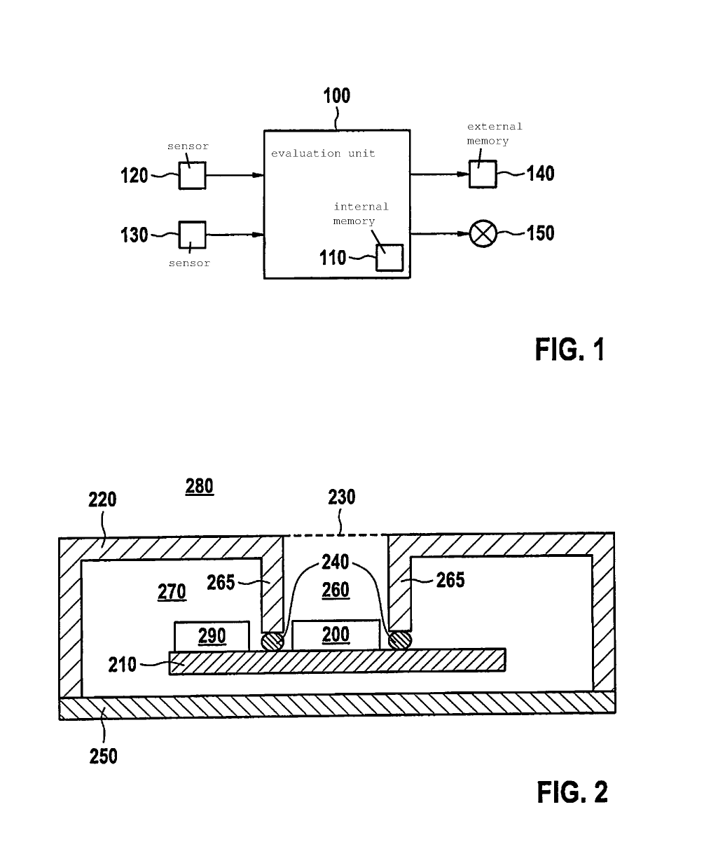

FIG. 1 explains the principle (of operation) of an example embodiment of the present invention with the aid of a block diagram. Using suitable sensors 120 and 130, an evaluation unit 100 detects humidity sensor variables and temperature sensor variables of the air in a detection volume. For this purpose, sensors 120 and 130 may be realized separately of one another or may be accommodated in a common humidity sensor element. On the basis of these sensor variables or their change over time, evaluation unit 100 is able to infer the humidity of the air in the detection volume. The air humidity or the ascertained air humidity variable may be stored in an internal memory 110 or an external memory 140 for further processing or later inspection. It is also possible to indicate the air humidity using a suitable indicator 150. This indicator may also be developed in the form of an acoustic indicator.

Another exemplary embodiment may provide for evaluation unit 100 to compare the ascertained air humidity variables to one or multiple threshold values. If the air humidity variable exceeds or falls below one or multiple threshold values, a warning signal may be produced, stored and/or indicated. Optionally, there may also be a provision for the warning signal to be produced only if the air humidity variable exceeds or falls below the threshold value(s) over a specified time period or multiple times.

One possible development of the construction of the humidity sensor is shown in FIG. 2. The humidity sensor in this instance is made of a housing, e.g., a cap 220 and a housing bottom 250. A recess 260 is developed in cap 220. In the present exemplary embodiment, this recess 260 runs in cylindrical form from the (outer) surface of cap 220 into the interior of the housing. For this purpose, walls 265 are provided laterally from the recess. Humidity sensor element 200 is provided on the bottom of recess 260, which detects in addition to the humidity (sensor) variable also the temperature variable of the air contained in the detection volume of the recess. As shown in FIG. 2, humidity sensor element 200 may be situated on a mounting board 210 that terminates recess 260. To secure the interior 270 of housing 220 and 250, walls 265 and mounting board 210 are connected to one another via a seal 240. Seal 240 may be implemented via a sealing ring. For defining the detection volume, a membrane 230 is provided on the upper end of recess 260, e.g. as an extension of the surface of cap 220. This membrane 230 is developed so as to allow for an exchange of air between the exterior 280 and the detection volume, while preventing the ingress of water. The detection volume is advantageously designed to be so small and/or to be situated in such close proximity to the surface of cap 220 that a quick exchange of the air between exterior 280 and recess 260 is possible. A development is particularly advantageous in which the humidity sensor element 200 occupies a substantial portion of recess 260, at least, however, one quarter of the detection volume.

Another specific embodiment may provide for also mounting evaluation unit 290 for evaluating the sensor signals on mounting board 210 of humidity sensor element 200. In order to protect evaluation unit 290 against external influences, the present invention provides for mounting board 290 to be attached in such a way that it projects with evaluation unit 290 into the interior 270 of housing 220 and 250. Thus it is possible for example to situate the seal between mounting board 210 and walls 265, between evaluation unit 290 and humidity sensor element 200, as shown in FIG. 2.

Another exemplary embodiment (not shown) may provide for the recess to extend through the housing so as to allow for an air flow through the housing. In this instance, the humidity sensor element may be situated on the walls of the recess or duct. Alternatively, there may also be a provision for the humidity sensor element to be situated, similarly as in FIG. 2, on a mount in the recess or duct. For this purpose, however, it is necessary that the mount be perforated so as to allow for an air flow through the housing.

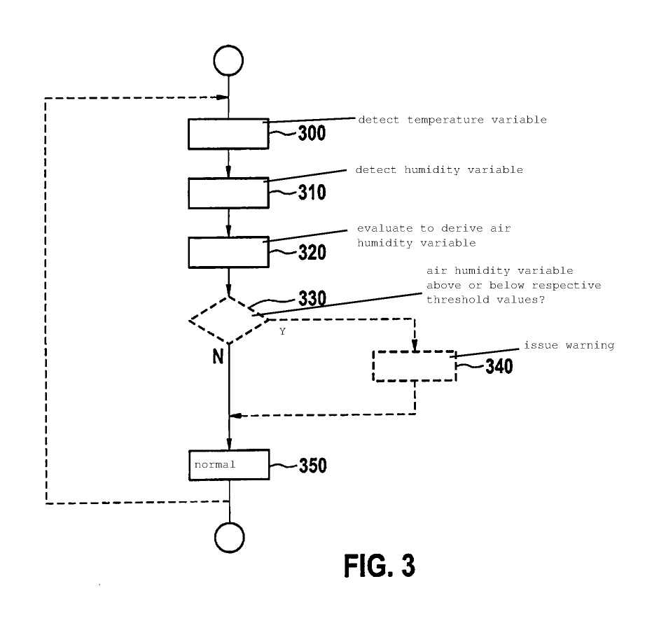

A method as shown in the flowchart of FIG. 3 maybe used to evaluate the sensor variables of the humidity sensor element. For this purpose, suitable sensors first detect a temperature variable in a step 300 and a humidity variable in a step 310. Both variables should be detected approximately at the same time or simultaneously since they are to be assigned to a common state for later evaluation. Subsequently, the detected sensor variables are evaluated in step 320 for deriving an air humidity variable. This air humidity variable may be stored in a subsequent step 350 before the evaluation method is terminated or is run through anew with the detection of further temperature and humidity variables.

In another variant of the present invention, the ascertainment of the air humidity variable in step 320 may also be performed as a function of the relative change of the detected sensor variables.

Optionally, there may be a provision for comparing the ascertained air humidity variable to one or more threshold values in a step 330. If it is detected that the air humidity is above a defined first threshold value, a warning signal may be produced, stored and/or indicated in a subsequent step 340. Alternatively, step 340 may also be run through if the air humidity falls below a defined second threshold value. Otherwise the method is normally run through with step 350.

The comparison in step 330 may also be performed over a specified time period, i.e., it may be determined to what extent the air humidity lies above/below a threshold value during a specified time period. For this purpose, the method is able to make use of stored data or may perform an indexing of the memory entry in the event that a respective threshold value is exceeded/undershot. It is furthermore possible to sum up the exceedance/undershooting of the threshold value. It is possible to perform the storage/indication of the warning signal only in the event of a sufficient frequency or duration of the exceedance/undershooting.

What is claimed is:

1. A humidity sensor, comprising:
a humidity sensor element; and
a housing having a recess, in which the humidity sensor element is situated, at least one portion of the recess being covered by a membrane that is at least permeable to humidity, but not permeable to water.

2. The humidity sensor as recited in claim 1, wherein the walls of the recess are formed by a material that cannot absorb humidity and also cannot influence the humidity content of the recess, the walls of the recess one of: (i) made of metal, or (ii) at least having an outer metal coating.

3. The humidity sensor as recited in claim 1, wherein the membrane defines a detection volume in the recess, which is in contact with an external volume at least partially with respect to the air humidity via the humidity-permeable membrane, wherein the membrane is to the greatest possible extent impermeable to water.

4. The humidity sensor as recited in claim 1, wherein the humidity sensor element occupies a substantial portion of the volume in the recess.

5. The humidity sensor as recited in claim 1, wherein the volume, which is bounded by the recess and the humidity-permeable membrane, maximally corresponds to four times the volume of the humidity sensor element.

6. The humidity sensor as recited in claim 1, wherein the recess is in the form of a cylinder, the humidity sensor element being provided on the bottom of the cylinder and the membrane being provided on the upper end of the cylinder, across from the humidity sensor element.

7. The humidity sensor as recited in claim 6, wherein the humidity sensor element is situated on a mounting board, which is connected to the walls of the cylindrical recess via a sealing ring.

8. The humidity sensor as recited in claim 7, wherein an evaluation unit is provided for the humidity sensor element, which is situated within the housing on the mounting board.

9. A method for evaluating a humidity sensor element, the method comprising:
detecting mutually associated temperature variables representing a temperature in surroundings of the humidity sensor element, and humidity variables of the humidity sensor element representing air humidity detected on the humidity sensor element; and
ascertaining an air humidity variable as a function of the change over time of the mutually associated temperature variables and humidity variables.

10. A data logger, comprising:
a humidity sensor a humidity sensor element, and a housing having a recess, in which the humidity sensor element is situated, at least one portion of the recess being covered by a membrane that is at least permeable to humidity, but not permeable to water; and
an evaluation unit which detects mutually associated temperature variables representing a temperature in surroundings of the humidity sensor element, and humidity variables of the humidity sensor element representing air humidity detected on the humidity sensor element, and ascertains an air humidity variable as a function of the change over time of the mutually associated temperature variables and humidity variables, wherein the ascertained air humidity variables being stored in a memory.

11. The data logger as recited in claim 10, further comprising:
an indicator element controlled as a function of a comparison of at least one ascertained air humidity variable with a threshold value.
